(19) United States
(12) Patent Application Publication
(10) Pub. No.: US 2016/0089268 A1
(43) Pub. Date: Mar. 31, 2016

(54) PHACOEMULSIFICATION HAND PIECE WITH INTEGRATED VENTURI ASPIRATION PUMP

(71) Applicant: NOVARTIS AG, Basel (CH)

(72) Inventors: JAMES Y. CHON, IRVINE, CA (US); JOHN ZHONGYU YAN, IRVINE, CA (US)

(21) Appl. No.: 14/501,997

(22) Filed: Sep. 30, 2014

(51) Int. Cl. A61F 9/007

(52) U.S. Cl. CPC A61F 9/00745

(57) ABSTRACT

An ophthalmic surgical hand piece comprises a driver coupled to a horn, a venturi pump with an inlet aspiration port and an outlet aspiration port, and a hand piece shell at least partially enclosing the driver, the horn, and the venturi pump. A needle is coupled to an end of the horn. A rigid, non-compliant aspiration path extends between the hollow needle and the outlet aspiration port. An aspiration pressure sensor measures a pressure in the aspiration path.

### BACKGROUND OF THE INVENTION

The present invention relates to phacoemulsification surgery and more particularly to a device that better regulates pressure experienced in the eye during cataract surgery.

The human eye functions to provide vision by transmitting light through a clear outer portion called the cornea, and focusing the image by way of a crystalline lens onto a retina. The quality of the focused image depends on many factors including the size and shape of the eye, and the transparency of the cornea and the lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. A typical surgical hand piece suitable for phacoemulsification procedures consists of an ultrasonically driven phacoemulsification hand piece, an attached hollow cutting needle surrounded by an irrigating sleeve, and an electronic control console. The hand piece assembly is attached to the control console by an electric cable and flexible tubing. Through the electric cable, the console varies the power level transmitted by the hand piece to the attached cutting needle. The flexible tubing supplies irrigation fluid to the surgical site and draws aspiration fluid from the eye through the hand piece assembly.

The operative part in a typical hand piece is a centrally located, hollow resonating bar or horn directly attached to a set of piezoelectric crystals. The crystals supply the required ultrasonic vibration needed to drive both the horn and the attached cutting needle during phacoemulsification, and are controlled by the console. The crystal/horn assembly is suspended within the hollow body or shell of the hand piece by flexible mountings. The hand piece body terminates in a reduced diameter portion or nosecone at the body's distal end. Typically, the nosecone is externally threaded to accept the hollow irrigation sleeve, which surrounds most of the length of the cutting needle. Likewise, the horn bore is internally threaded at its distal end to receive the external threads of the cutting tip. The irrigation sleeve also has an internally threaded bore that is screwed onto the external threads of the nosecone. The cutting needle is adjusted so that its tip projects only a predetermined amount past the open end of the irrigating sleeve.

During the phacoemulsification procedure, the tip of the cutting needle and the end of the irrigation sleeve are inserted into the anterior segment of the eye through a small incision in the outer tissue of the eye. The surgeon brings the tip of the cutting needle into contact with the lens of the eye, so that the vibrating tip fragments the lens. The resulting fragments are aspirated out of the eye through the interior bore of the cutting needle, along with irrigation solution provided to the eye during the procedure, and into a waste reservoir.

Throughout the procedure, irrigating fluid is pumped into the eye, passing between the irrigation sleeve and the cutting needle and exiting into the eye at the tip of the irrigation sleeve and/or from one or more ports, or openings, cut into the irrigation sleeve near its end. This irrigating fluid is critical, as it prevents the collapse of the eye during the removal of the emulsified lens. The irrigating fluid also protects the eye tissues from the heat generated by the vibrating of the ultrasonic cutting needle. Furthermore, the irrigating fluid suspends the fragments of the emulsified lens for aspiration from the eye.

A common phenomenon during a phacoemulsification procedure arises from the varying flow rates that occur throughout the surgical procedure. Varying flow rates result in varying pressure losses in the irrigation fluid path from the irrigation fluid supply to the eye, thus causing changes in pressure in the anterior chamber (also referred to as Intra-Ocular Pressure or IOP.) Higher flow rates result in greater pressure losses and lower IOP. As IOP lowers, the operating space within the eye diminishes.

Another common complication during the phacoemulsification process arises from a blockage, or occlusion, of the aspirating needle. As the irrigation fluid and emulsified tissue is aspirated away from the interior of the eye through the hollow cutting needle, pieces of tissue that are larger than the diameter of the needle's bore may become clogged in the needle's tip. While the tip is clogged, vacuum pressure builds up within the tip. The resulting drop in pressure in the anterior chamber in the eye when the clog is removed is known as post-occlusion surge. This post-occlusion surge can, in some cases, cause a relatively large quantity of fluid and tissue to be aspirated out of the eye too quickly, potentially causing the eye to collapse and/or causing the lens capsule to be torn.

Various techniques, such as venting the aspiration line, have been designed to reduce this surge. However, there remains a need for improved phacoemulsification devices that reduce post-occlusion surge as well as maintain a stable IOP throughout varying flow conditions.

### SUMMARY OF THE INVENTION

In one embodiment of the present invention, an ophthalmic surgical hand piece comprises a driver coupled to a horn; a venturi pump with an inlet aspiration port and an outlet aspiration port; and a hand piece shell at least partially enclosing the driver, the horn, and the venturi pump. A hollow needle is removably coupled to the horn at an end of the horn. A rigid, non-compliant aspiration path extends between the hollow needle and the outlet aspiration port. The rigid, non-compliant aspiration path may be made of titanium, stainless steel, or other similar material. An aspiration pressure sensor is located within the hand piece shell. The aspiration pressure sensor measures a pressure in the aspiration path, and the measured pressure is used to control the venturi pump. The measured pressure indicates one of an occlusion or occlusion break, and the venturi pump is controlled to compensate for one of the occlusion or occlusion break. An outlet aspiration port and a compressed air port are located at a proximal end of the hand piece shell. The outlet aspiration port and the compressed air port are coupled to the venturi pump. An inlet aspiration port is coupled to the venturi pump. The inlet aspiration port is enclosed by the hand piece shell. The outlet aspiration port is configured to be coupled to an aspiration line, and the compressed air port is configured to be coupled to a compressed air line.

In another embodiment of the present invention, an ophthalmic surgical hand piece comprises a driver coupled to a horn; a hollow needle removably coupled to the horn at an end of the horn; a venturi pump with an inlet aspiration port and an outlet aspiration port; a rigid, non-compliant aspiration path extending between the hollow needle and the outlet aspiration port; and a hand piece shell at least partially enclosing the driver, the horn, and the venturi pump. An aspiration pressure sensor is located within the hand piece shell. The aspiration pressure sensor measures a pressure in the aspiration path. The measured pressure can be used to control the venturi pump. The hand piece may also comprise: an outlet aspiration port and a compressed air port located at a proximal end of the hand piece shell, the outlet aspiration port and the compressed air port coupled to the venturi pump; and an inlet aspiration port coupled to the venturi pump, the inlet aspiration port enclosed by the hand piece shell. The outlet aspiration port is configured to be coupled to an aspiration line and the compressed air port is configured to be coupled to a compressed air line.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the components in the fluid path of a phacoemulsification system including a hand piece with an integrated venture aspiration pump according to the principles of the present invention.

FIG. 2 is a block diagram of a phacoemulsification hand piece with an integrated aspiration pump according to the principles of the present invention.

FIG. 3 is a block diagram of a phacoemulsification hand piece with an integrated aspiration pump according to the principles of the present invention.

FIG. 4 is a side cross-section view of an embodiment of an integrated venturi pump according to the principles of the present invention.

FIG. 5 is a diagram of an embodiment of a phacoemulsification hand piece according to the principles of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

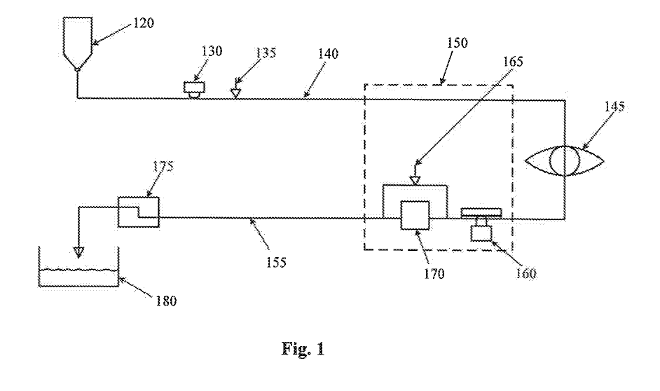

FIG. 1 is a diagram of the components in the fluid path of a phacoemulsification system including a hand piece with an integrated venture aspiration pump according to the principles of the present invention. FIG. 1 depicts the fluid path through the eye 145 during cataract surgery. The components include an irrigation source 120, an optional irrigation pressure sensor 130, an optional irrigation valve 135, an irrigation line 140, a hand piece 150, an aspiration line 155, an optional aspiration pressure sensor 160, an optional vent valve 165, a venturi pump 170, a reservoir 175 and a drain bag 180. The irrigation line 140 provides irrigation fluid to the eye 145 during cataract surgery. The aspiration line 155 removes fluid and emulsified lens particles from the eye during cataract surgery.

When irrigation fluid exits irrigation source 120, it travels through irrigation line 140 and into the eye 145. An irrigation pressure sensor 130 measures the pressure of the irrigation fluid in irrigation line 140. An optional irrigation valve 135 is also provided for on/off control of irrigation. Irrigation pressure sensor 130 is implemented by any of a number of commercially available fluid pressure sensors.

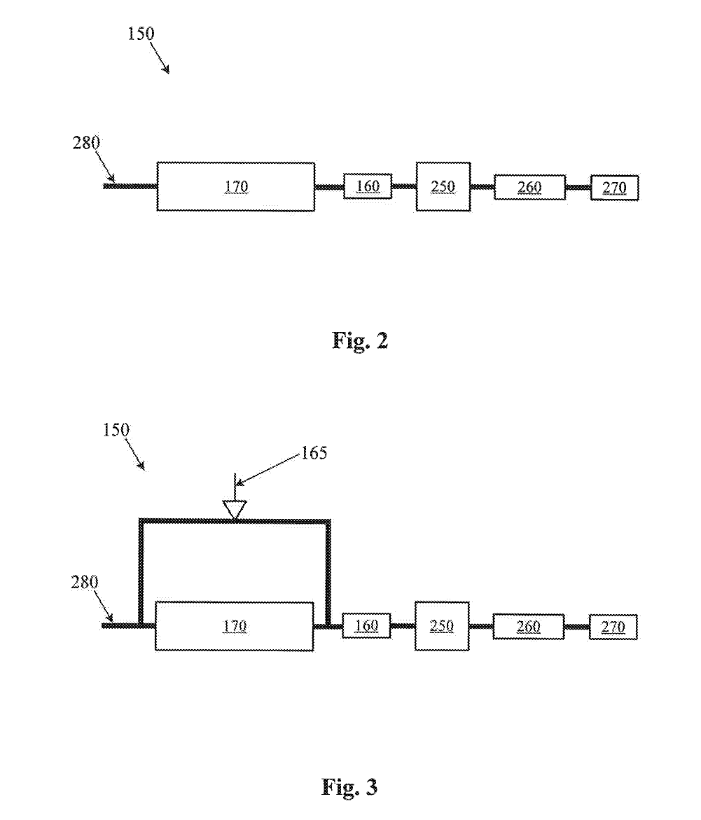

A hand piece 150 is placed in relation to the eye 145 during a phacoemulsification procedure. The hand piece 150 has a hollow needle (270 in FIGS. 2 & 3) that is ultrasonically vibrated in the eye to break up the diseased lens. A sleeve located around the needle provides irrigation fluid from irrigation line 140. The irrigation fluid passes through the space between the outside of the needle and the inside of the sleeve. Fluid and lens particles are aspirated through the hollow needle. In this manner, the interior passage of the hollow needle is fluidly coupled to aspiration line 155. Venturi pump 170 draws the aspirated fluid from the eye 145. An optional aspiration pressure sensor 160 measures the pressure in the aspiration line. An optional vent valve can be used to vent the vacuum created by pump 170. The aspirated fluid passes through reservoir 175 and into drain bag 180.

FIG. 2 is a block diagram of a phacoemulsification hand piece with an integrated venturi pump according to the principles of the present invention. In FIG. 2, hand piece 150 comprises venturi pump 170, optional aspiration pressure sensor 160, driver 250, horn 260, needle 270, and aspiration line 280. Venturi pump 170 is located in series with and draws fluid through aspiration line 280. Optional aspiration pressure sensor 160 is located between venture pump 170 and the eye 145. Driver 250 vibrates horn 260 which in turn vibrates needle 270.

In FIG. 2, Aspiration line 280 is fluidly coupled to ventui pump 170. Aspiration line also extends through or around drive 250, horn 260, and needle 270. A lumen in needle 270 is fluidly coupled to aspiration line 280. As described above, fluid and lens particles are aspirated through the lumen of needle 270. Venturi pump 170 draws fluid and lens particles through the lumen of needle 270.

Driver 250 is typically an ultrasonic driver that produces ultrasonic vibrations in horn 260. Horn 260 is typically a mass of metal that is coupled to driver 250 and needle 270. In this manner, vibrations produced by driver 250 are transferred to horn 260 and to needle 270. Needle 270 is placed in the eye and vibrated to fragment a cataractous lens.

Aspiration pressure sensor 160 measures the aspiration pressure in aspiration line 280. While shown as located between venture pump 170 and driver 250, aspiration pressure sensor 160 may be located at any location between pump 170 and the eye 145. Aspiration pressure sensor 160 may be implemented by any of a number of known pressure sensor devices.

FIG. 3 is a block diagram of a phacoemulsification hand piece with an integrated aspiration pump according to the principles of the present invention. The example of FIG. 3 has the elements of FIG. 2 plus an optional vent valve 165. When optional vent valve 165 is present, it acts to provide a venting path for the venturi pump 170. In this manner, venturi pump 170 can be vented, for example, to atmosphere when vent valve 165 is opened. Alternatively, venturi pump 170 can be vented to the irrigation line 140. As shown in FIG. 3, aspiration line 280 has two paths—one path that goes through venturi pump 170, and another path that bypasses venturi pump 170. When vent valve 165 is opened, the aspiration or vacuum produced by pump 170 is decreased as a result of it being vented to atmosphere.

The control of aspiration vacuum can be based on a reading from aspiration pressure sensor 160. Aspiration pressure sensor 160 is located between the pump and the eye. In this manner, aspiration pressure sensor 160 accurately reads the pressure conditions in the aspiration line very close to the eye. Such a reading can be used to precisely control the aspiration vacuum that is applied to the eye.

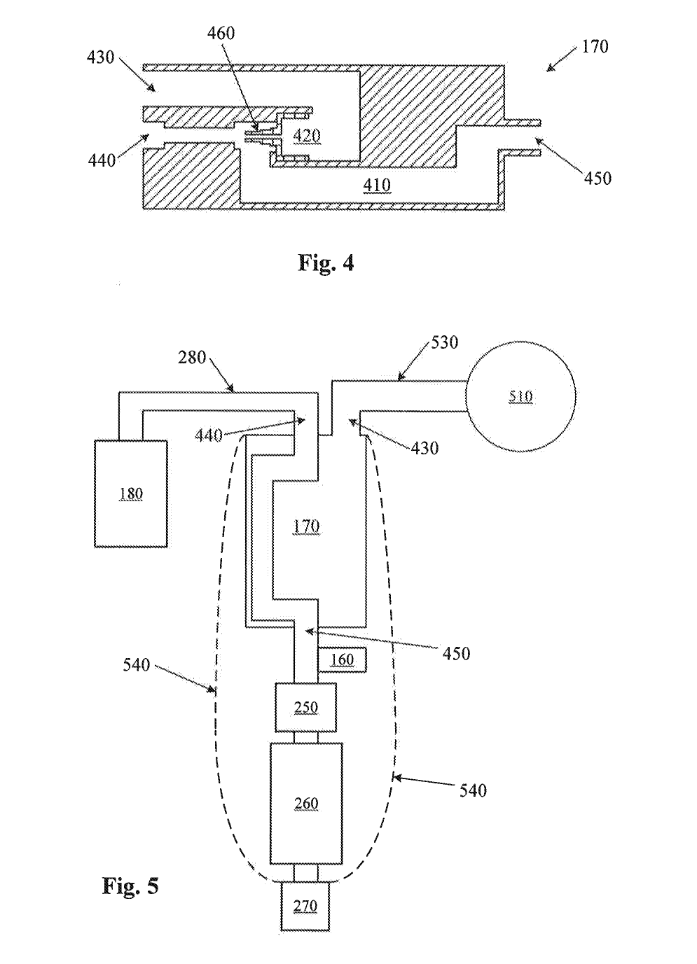

FIG. 4 is a side cross-section view of an embodiment of venturi pump 170 according to the principles of the present invention. In the example of FIG. 4, venturi pump 170 may be integrated into a phacoemulsification hand piece. In FIG. 4, venturi pump 170 has a venturi element 460 located adjacent to a venturi chamber 420. Compressed air port 430 is coupled to venturi chamber 420 and provides compressed gas (such as compressed air) to venturi element 460. An inlet aspiration port 450 is coupled to an outlet aspiration port 440 via a lumen 410.

In operation, venturi pump 170 produces a vacuum that draws fluid into inlet aspiration port 450, through lumen 410, and out of outlet aspiration port 440. Compressed air is provided to venturi element 460 via compressed air port 430 to produce this vacuum.

FIG. 5 is a diagram of an embodiment of a phacoemulsification hand piece according to the principles of the present invention. In the example of FIG. 5, a hand piece shell 540 partially or fully encloses venturi pump 170, aspiration pressure sensor 160, driver 250, and horn 260. A needle 270 is coupled to horn 260. A pressurized gas source 510 provides pressurized gas (such as compressed air) through compressed air line 530 to venturi pump 170. An aspiration line 280 extends from outlet aspiration port 440 to drain bag 180.

A continuous aspiration lumen is present though a hollow in needle 270, through or around horn 260, through or around driver 250, through venturi pump 170, and through aspiration line 280. Fluid and lens particles can be aspirated through this continuous aspiration lumen and into drain bag 180 during cataract surgery.

Hand piece shell 540 may be made of stainless steel, titanium or other durable and sterilizable material. Typically, hand piece shell 540 is configured to be grasped and manipulated by a surgeon during cataract surgery. In the example of FIG. 5, hand piece shell 540 fully enclosed venturi pump 170, aspiration pressure sensor 160, driver 250, and horn 260. Hand piece shell 540 may be sealed on both ends. Compressed air port 430 and outlet aspiration port 440 extend from one end of hand piece shell 540. Needle 270 is removably attached to the other end of hand piece shell 540.

In this example, aspiration pressure sensor 160 measures a pressure in the aspiration path near inlet aspiration port 450. The aspiration path between inlet aspiration port 450 and the tip end of needle 270 is typically very short and made of a solid rigid material such as stainless steel or titanium tubing. As such, aspiration pressure sensor 160 is capable of reading a pressure that is identical or very close to the pressure in the eye during surgery. The pressure reading from aspiration pressure sensor 160 can be used to control the operation of venturi pump 170 during surgery. A pressure reading from aspiration pressure sensor 160 may be used to control operation of venturi pump 160, for example, by sensing an increase in pressure from an occlusion and a decrease in pressure from occlusion break and altering the vacuum produced by venturi pump 170. When an increased pressure is sensed by aspiration pressure sensor 160, venturi pump 170 may be controlled so as to prevent a surge during occlusion break. When a sudden decrease in pressure is sensed by aspiration pressure sensor 160 (indicating occlusion break), venturi pump 170 may be controlled so as to lessen or prevent the surge of fluid caused by occlusion break.

The design of the present invention allows for the venturi pump 170 to be very close to the eye 145. The distance between the venturi pump 170 and the eye 145 can be made to be very small—on the order of inches. Placing the venturi pump 170 close to the eye 145 allows for a very short aspiration path to be located between the venturi pump 170 and the eye 145. Moreover, the aspiration path located between the venturi pump 170 and the eye 145 can be rigid (for example, it can be made of titanium or stainless steel). This short length of non-compliant material that makes up the aspiration path between the venturi pump 170 and the eye 145 largely eliminates the surge effect associated with conventional phacoemulsification systems.

In conventional phacoemulsification systems, the aspiration pump is located in a console. A relatively long length of flexible tubing (six feet or more) is located between the aspiration pump and the eye. This relatively long length of flexible tubing has a lot of compliance—it can stretch in response to changes in vacuum pressure. This compliance results in surges as previously described. By incorporating the venturi pump 170 in the hand piece 150 (and thereby placing it very close to the eye) and having a very short, non-compliant aspiration path between the venturi pump 170 and the eye 145, these surges can be eliminated, thus resulting in a safer and more efficient surgery.

From the above, it may be appreciated that the present invention provides an improved hand piece for phacoemulsification surgery. The present invention provides a device that more precisely controls fluid pressure and/or flow during surgery. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ophthalmic surgical hand piece comprising:
a driver coupled to a horn;
a venturi pump with an inlet aspiration port and an outlet aspiration port; and
a hand piece shell at least partially enclosing the driver, the horn, and the venturi pump.

2. The hand piece of claim 1 further comprising:
a hollow needle removably coupled to the horn at an end of the horn.

3. The hand piece of claim 2 further comprising:
a rigid, non-compliant aspiration path extending between the hollow needle and the outlet aspiration port.

4. The hand piece of claim 3 further comprising:
an aspiration pressure sensor located within the hand piece shell.

5. The hand piece of claim 4 wherein the aspiration pressure sensor measures a pressure in the aspiration path.

6. The hand piece of claim 5 wherein the measured pressure is used to control the venturi pump.

7. The hand piece of claim 6 wherein the measured pressure indicates one of an occlusion or occlusion break, and the venturi pump is controlled to compensate for one of the occlusion or occlusion break.

8. The hand piece of claim 3 wherein the rigid, non-compliant aspiration path is made of a material selected from the group comprising: titanium and stainless steel.

9. The hand piece of claim 1 further comprising:
an outlet aspiration port and a compressed air port located at a proximal end of the hand piece shell, the outlet aspiration port and the compressed air port coupled to the venturi pump; and
an inlet aspiration port coupled to the venturi pump, the inlet aspiration port enclosed by the hand piece shell.

10. The hand piece of claim 9 wherein the outlet aspiration port is configured to be coupled to an aspiration line and the compressed air port is configured to be coupled to a compressed air line.

11. An ophthalmic surgical hand piece comprising:
a driver coupled to a horn;
a hollow needle removably coupled to the horn at an end of the horn;
a venturi pump with an inlet aspiration port and an outlet aspiration port;
a rigid, non-compliant aspiration path extending between the hollow needle and the outlet aspiration port; and
a hand piece shell at least partially enclosing the driver, the horn, and the venturi pump.

12. The hand piece of claim 11 further comprising:
an aspiration pressure sensor located within the hand piece shell.

13. The hand piece of claim 12 wherein the aspiration pressure sensor measures a pressure in the aspiration path.

14. The hand piece of claim 13 wherein the measured pressure is used to control the venturi pump.

15. The hand piece of claim 14 wherein the measured pressure indicates one of an occlusion or occlusion break, and the venturi pump is controlled to compensate for one of the occlusion or occlusion break.

16. The hand piece of claim 11 wherein the rigid, non-compliant aspiration path is made of a material selected from the group comprising: titanium and stainless steel.

17. The hand piece of claim 11 further comprising:
an outlet aspiration port and a compressed air port located at a proximal end of the hand piece shell, the outlet aspiration port and the compressed air port coupled to the venturi pump; and
an inlet aspiration port coupled to the venturi pump, the inlet aspiration port enclosed by the hand piece shell.

18. The hand piece of claim 17 wherein the outlet aspiration port is configured to be coupled to an aspiration line and the compressed air port is configured to be coupled to a compressed air line.
